(19) United States
(12) Patent Application Publication
(10) Pub. No.: US 2026/0248949 A1
(43) Pub. Date: Aug. 27, 2026

(54) COMPOSITION AND METHOD

(71) Applicant: Optimus Salvus Limited, Gibraltar (GI)

(72) Inventors: Peter Joseph STABLER, Glasgow (GB); Gregory Alan STOLOFF, London (GB)

(21) Appl. No.: 18/870,739

(22) PCT Filed: Jun. 1, 2023
(86) PCT No.: PCT/GB2023/051452

§ 371 Date: Dec. 2, 2024

(30) Foreign Application Priority Data
Jun. 1, 2022 (GB) 2208130.1

(51) Int. Cl. A61K 47/69; A61K 31/00; A61K 31/07; A61K 31/122; A61K 31/355; A61K 31/366; A61K 31/59

(52) U.S. Cl. CPC A61K 47/69; A61K 31/07; A61K 31/122; A61K 31/355; A61K 31/366; A61K 31/59; A61K 31/658

(57) ABSTRACT

The present invention relates to a solid composition comprising a lipophilic drug (such as a cannabinoid or Vitamin E) and a cyclodextrin wherein the lipophilic drug and the cyclodextrin are in the form of a combined complexation, and the lipophilic drug and the cyclodextrin in the combined complexation are either at a molar ratio of about 1:2; and methods of obtaining the same.

### FIELD OF INVENTION

The present invention relates to solid compositions comprising lipophilic drug and cyclodextrin, methods of obtaining the same, formulation of same, and therapeutic and non-therapeutic uses thereof.

### BACKGROUND OF THE INVENTION

Enhancing the solubility of poorly-water soluble lipophilic drugs represents an ongoing challenge in drug formulation.

Lipophilic drugs (particularly those of certain logP values above 1) are known to have poor solubility in aqueous media. The use of such drugs as therapeutic agents presents many challenges owing to their poor solubility which results in poor bioavailability and hence decreased efficacy and increased side effects.

For instance, cannabinoids represent one class or group of lipophilic drugs that are poorly water soluble.

Cannabinoids are lipophilic compounds found in cannabis. The most notable cannabinoid is the phytocannabinoid tetrahydrocannabinol (THC) (Delta9-THC or Delta8-THC), the primary psychoactive compound in cannabis. Cannabidiol (CBD) is another major constituent of the plant.

Certain cannabinoids may be useful in the treatment of various medical conditions such as pain, chronic pain, inflammation, alcoholism, drug addiction, depression, post-traumatic stress disorder, anxiety, multiple sclerosis, epilepsy Parkinson's disease. Cannabinoids may also be useful in ameliorating the side effects of chemotherapy.

The endocannabinoid system (ECS) regulates many functions of the human body. The ECS plays an important role in multiple aspects of neural functions, including the control of movement and motor coordination, learning and memory, emotion and motivation, addictive-like behaviour and pain modulation, among others.

The ECS is a biological system composed of endocannabinoids, which are endogenous lipid-based retrograde neurotransmitters that bind to cannabinoid receptors (CBRs), and cannabinoid receptor proteins that are expressed throughout the vertebrate central nervous system (including the brain) and peripheral nervous system.

Two primary cannabinoid receptors (CBRs) have been identified, CB1 and CB2. Cannabidiol (CBD) is a naturally occurring cannabinoid which mainly binds to CB2 receptors and is an allosteric inhibitor of CB1 receptors.

An appropriate administration route is key to obtaining the desired therapeutic effect. For example, cannabinoids have been formulated for oral administration by dissolving in alcohols—e.g. the aerosolised mist containing Nabiximols for the relief of neuropathic pain, which spray contains approximately 1:1 ratio of CBD and THC dissolved in a mixture of ethanol and propylene glycol. Cannabinoids can be formulated for administration in an injectable form by dissolving in oils or fats.

However, there are many drawbacks of such formulations, such as the fact that fats and oils are challenging to sterilise. As such, increasing the solubility of cannabinoids would open up many more forms of administration of the cannabinoid in a safe and cost effective manner.

Therefore, there is a need for new formulations of poorly soluble lipophilic drugs such as cannabinoids (e.g. CBD) and certain vitamins (e.g. vitamin E), in order to increase the solubility in aqueous medium, thereby increasing absorption, bioavailability and hence, efficacy of the drug. Preferably, the process for obtaining such a new formulation is low cost and the product obtained is stable without containing toxic excipients.

### SUMMARY OF THE INVENTION

The present invention seeks to address the problem outlined above by providing a solid composition comprising lipophilic drug and cyclodextrin, such as cannabinoid and cyclodextrin. This is achieved by a process described herein which allows for fine control over the complexation process and, importantly, results in largely complete complexation of the lipophilic drug. Existing cyclodextrin complexation processes do not provide for this level of control and so, while improvements in solubility of the lipophilic drug may be observed using such approaches, even at apparently significant levels, the product is generally a mixture of partly complexed material, cyclodextrin starting material and uncomplexed drug. The improved solubility is therefore more an uplift based on the amount of lipophilic drug which is complexed but is suboptimal in that not all available cyclodextrin has been complexed with drug or, put another way, not all lipophilic drug introduced to the system has been complexed.

The process described herein allows for substantially all lipophilic drug introduced into the system to be complexed with the cyclodextrin and this can, in embodiments, provide an improvement in water solubility of the complexed drug versus the uncomplexed form which is orders of magnitude greater than that achieved in the prior art.

As will be evident from the data presented below, compositions comprising a lipophilic drug, such as a cannabinoid, and a cyclodextrin, according to the present invention may have significantly improved solubility and/or bioavailability compared to similar compositions comprising plain (i.e. ‘non-complexed’, or ‘uncomplexed’ with cyclodextrin) lipophilic drugs, such as cannabinoids.

Accordingly, a first aspect of the invention is a solid composition comprising lipophilic drug and cyclodextrin wherein the lipophilic drug and the cyclodextrin are in the form of a combined complexation and the lipophilic drug and the cyclodextrin in the combined complexation are at a molar ratio of about 1:2.

A second aspect of the invention is the solid composition according to the first aspect for use in the treatment of a disease, disorder or condition.

A third aspect of the invention is a method of treatment of a disease, disorder or condition comprising administering to a subject the solid composition of the first aspect of the invention.

A fourth aspect of the invention is the use of the solid composition of the first aspect of the invention in the manufacture of a medicament for the treatment of a disease, disorder or condition.

A fifth aspect of the invention is the non-therapeutic use of the solid composition of the first aspect of the invention.

A sixth aspect of the invention is a process of making a solid composition comprising a lipophilic drug and a cyclodextrin, wherein:
the lipophilic drug and the cyclodextrin are in the form of a combined complexation at a molar ratio of about 1:2,
   wherein the process comprises the steps of:
a. providing the lipophilic drug;
b. providing the cyclodextrin;
c. combining the lipophilic drug and the cyclodextrin in the presence of water to form a composition;
d. mixing the composition until the combined complexation of lipophilic drug and cyclodextrin precipitates;
e. collecting the precipitate of step d. to provide the solid composition comprising the lipophilic drug and the cyclodextrin in the form of a combined complexation.

A seventh aspect of the invention is the solid composition obtainable by the process of the sixth aspect of the invention.

An eighth aspect of the invention is the solid composition of the first aspect when produced by the process of the sixth aspect.

### DESCRIPTION OF THE FIGURES

FIG. 1 shows the aqueous solubility of cyclodextrin (CD) in the presence of CBD.

FIG. 2 shows the solubility of CD in the presence of tocopherol succinate.

FIG. 3 shows aqueous CD solubility in the presence of increasing amounts of CBD. As the complex forms, the concentration of CD decreases, then plateaus until the complex fully forms (at about 1:2 CBD:CD).

FIGS. 4(a) to 4(c) show DSC analyses of CBD, CD raw materials and a 2:1 beta-CD:CBD dimer complex, respectively.

FIG. 5 shows the solubility profile of a 2:1 beta-CD:CBD dimer, upon heating and cooling, using UV spectroscopy.

FIG. 6 shows a phase solubility diagram for CBD/Cyclodextrin.

FIG. 7 shows the solubility of CD in the presence of CBG.

### DETAILED DESCRIPTION OF THE INVENTION

According to the first aspect of the invention, there is provided a solid composition for oral administration comprising lipophilic drug and cyclodextrin wherein the lipophilic drug and the cyclodextrin are in the form of a combined complexation, and the lipophilic drug and the cyclodextrin in the combined complexation are at a molar ratio of about 1:2.

The combined complexation at a molar ratio of about 1:2 may provide particular benefits, in terms of improving the solubility; and/or release profile; and/or bioavailability; and/or stability; and/or formulation for delivery; of the lipophilic drug; and/or the commercial viability of the complexation process and complexed product; and/or in optimising the amount of lipophilic drug complexed within a batch; and/or the ability to complex certain lipophilic drugs.

Without wishing to be bound by theory, it is believed that the dimer complex is formed sequentially: first, the lipophilic drug, such as a cannabinoid, complexes with the cyclodextrin in a 1:1 molar ratio to form a monomer, then the monomer subsequently complexes with a further equivalent of cyclodextrin to form the dimer complex (i.e. molar ratio of lipophilic drug, such as a cannabinoid, to cyclodextrin of about 1:2).

Accordingly, when the composition comprising such a dimer complex is administered in vivo, it is believed that the dimer dissociates into one equivalent of monomer (i.e., the lipophilic drug, such as a cannabinoid, initially remains complexed to one cyclodextrin molecule) and one equivalent of free cyclodextrin. This provides the technical benefit of the complex maintaining solubility even after this partial dissociation and thus the lipophilic drug is not excreted and it can be absorbed into the bloodstream as either the monomer or dimer complex. Once absorbed into the bloodstream and diluted by blood plasma, more dimer dissociates into monomer; the monomer dissociates further releasing the lipophilic drug, such as a cannabinoid, thus full dissociation and release of the lipophilic drug into the bloodstream is achieved. On the contrary, if after administration into the body the dimer dissociated immediately into free cyclodextrin and free lipophilic drug prior to absorption into the bloodstream, the lipophilic drug would simply precipitate and be excreted and would therefore not be available for absorption. Therefore, the solubility and/or bioavailability of the lipophilic drug may be improved if it is administered in the form of the cyclodextrin dimer.

Additionally, in embodiments of the disclosure, the dimer has lower aqueous solubility compared to the monomer (i.e. lipophilic drug complexed with cyclodextrin in 1:1 molar ratio) which facilitates the preparation process, as the dimer precipitates selectively out of the aqueous reaction mixture. This may particularly be the case with the use of beta-cyclodextrin as the particular cyclodextrin in the complex, based on its relatively low water solubility versus the other native cyclodextrins. When other cyclodextrins are employed, such as alpha- or gamma-cyclodextrin, then it may be desirable to combine the lipophilic drug and cyclodextrin in an aqueous solvent. That is, water may be blended with another solvent, such as an alcohol, to modify the solubility of the combined complexation product accordingly, so that it is less than its equilibrium concentration.

The term “comprising” or variants thereof will be understood to imply the inclusion of a stated element, integer or step, or group of elements, integers or steps, but not the exclusion of any other element, integer or step, or group of elements, integers or steps. The term “comprising” or “comprises” will include references to the component consisting essentially of (such as consisting of) the relevant features.

The terms “consisting”, “consists” or variants thereof is to be understood to imply the inclusion of a stated element, integer or step, or group of elements, integers or steps, and the exclusion of any other element, integer or step or group of elements, integers or steps.

As used herein, “combined complexation” refers to a chemical complexation in which the complex has one or more chemical and physical properties that are different from the properties of the individual starting chemicals and from the properties of a mixture in which the chemicals are not interacting to form a complex. “Combined complexation” includes inclusion complexes, in which a chemical compound (“host”) has a cavity into which a “guest” compound can be accommodated. The combined complexation of lipophilic drug and cyclodextrin of the present disclosure is a complex in which the lipophilic drug is simultaneously accommodated or “hosted” within two cyclodextrin molecules (a 1:2 complex or “dimer”). “Combined complexation” does not include within its scope clathrates or those cyclodextrin-based complexes in which the cyclodextrin molecules are bridged, such as by a covalently bonded bridging moiety.

The term “lipophilic”, as used herein in relation to the drug to be complexed with the cyclodextrin, is to be construed broadly as a compound which has poor water solubility such that the drug requires complexation with a cyclodextrin to improve its solubility for formulation or in vivo delivery. In certain embodiments, the term may relate to a class II or class III compound as defined and measured under the biopharmaceutics classification system (BCS) which classifies drugs on the basis of both their solubility and permeability. In certain embodiments, the lipophilic drug may have a logP of between 3 to 9, preferably 4 to 8, more preferably 5 to 8, or 5 to 7. In certain embodiments, the lipophilic drug may have a size of between 100 g/mol to 700 g/mol, preferably between 200 g/mol to 600 g/mol. In certain embodiments, any two or more of the following statements may be true for the lipophilic drug: it may be a BCS class II or class III compound; and/or have a logP of between 100 g/mol to 700 g/mol, preferably between 200 g/mol to 600 g/mol; and/or have a logP of between 3 to 9, preferably 4 to 8, more preferably 5 to 8 including 5 to 7.

The terms “precipitate” or “precipitates”, as used herein in relation to the process of the sixth aspect wherein the combined complexation of lipophilic drug and cyclodextrin precipitates means that the combined complexation has formed in solution and has dropped out of solution as a solid combined complexation. While the reaction to form the combined complexation is still proceeding, preferably, both the lipophilic drug and cyclodextrin are at saturation in the solution and so during this period the precipitation of the combined complexation may not reduce the dissolved concentration of either starting materials, nor the complexation products, but is nonetheless, for the present purposes, considered to be a precipitate.

In the first aspect of the invention, the composition comprises a combined complexation between a lipophilic drug and a cyclodextrin.

Suitably the lipophilic drug and cyclodextrin form an inclusion complex, in which the “host” is cyclodextrin and the “guest” is the lipophilic drug.

Cyclodextrins (CDs) are a family of cyclic oligosaccharides, consisting of a macrocyclic ring of glucose subunits joined by α-1,4 glycosidic bonds. Cyclodextrins have been used in drug development to improve aqueous solubility, dissolution rate, and absorption of many lipophilic drugs. With a hydrophobic interior and hydrophilic exterior, cyclodextrins can, under appropriate conditions, form inclusion complexes with some known hydrophobic compounds.

However, until the present disclosure, it had not been realised that control over aspects of the process conditions when the combined complexation of lipophilic drug and cyclodextrin is being formed, could provide for significant additional benefits of the final solid composition of lipophilic drug and cyclodextrin combined complexation. Prior uses of CDs to improve the solubility of lipophilic compounds lack fine control and so, although an improvement in solubility is frequently observed for the inclusion complex versus uncomplexed or plain lipophilic compound, this improvement is not optimised and the complex is generally variable in quality/purity and lacking pharmaceutical grade reproducibility. For example, while cannabidiol (CBD) has been complexed with CDs previously and the improvement in solubility of the final product versus CBD alone in water may be as much as 10-fold, the process exemplified herein demonstrates unexpectedly that large further gains or orders of magnitude can be achieved. This is based upon achieving a much higher degree of complexation of the CBD in the complexation system and so a higher purity product. Further, it has been felt that some lipophilic compounds could not be successfully complexed with CDs but the approach described herein is shown to have significantly wider applicability than expected due to the understanding, and manipulation, of the underlying complexation dynamics.

In one embodiment, the cyclodextrin is selected from the group consisting of a native alpha-, beta-, gamma cyclodextrin, and methyl beta cyclodextrin; preferably, the cyclodextrin is native alpha-, beta-, or gamma cyclodextrin. More preferably, the cyclodextrin is native beta-cyclodextrin.

In another embodiment, the cyclodextrin is hydroxypropylated-beta-cyclodextrin or sulfobutylated-beta-cyclodextrin.

In a preferred embodiment of the disclosure, the cyclodextrin is native alpha-, beta-, or gamma cyclodextrin wherein ‘native’ means the cyclodextrin is unmodified. Such native cyclodextrins also exclude bridged cyclodextrins.

As is demonstrated herein, a range of different classes of lipophilic drugs/compounds can be complexed with CDs using the present process.

Suitably the lipophilic drug has a size that is smaller than the size of the cavity of the CD, such as beta-cyclodextrin. Preferably, the lipophilic drug is between 100 and 700 g/mol. More preferably, the lipophilic drug is between 200 and 600 g/mol, or between 300 and 600 g/mol, or between 300 and 550 g/mol, or between 250 and 550 g/mol.

In embodiments, the lipophilic drug is classed as Class II or III using the Biopharmaceutics Classification System (BCS).

Preferably, the lipophilic drug has at least one carbon chain which is at least 3-carbon atoms long such that when the lipophilic drug is in the combined complexation with the CD, the at least one 3-carbon long chain is in close proximity to the protons in the 3- and/or 5-position of the sugar rings of the CD cavity. More preferably, such lipophilic drug has a logP greater than or equal to 3, such as between 3 and 9, preferably between 3 and 8 and more preferably between 3 and 7.

In embodiments, the lipophilic drug is a compound having a phenolic ring which may be substituted and/or fused with a further ring. The phenolic ring or the further fused ring may be substituted with an alkyl or alkenyl chain. Preferably, the lipophilic drug has at least one 3-carbon long chain such that when the lipophilic drug is in the combined complexation with the CD, the at least one 3-carbon long chain is in close proximity to the protons in the 3- and/or 5-position of the sugar rings of the CD cavity.

Suitably the lipophilic drug is the only active agent in the composition.

Preferably, the lipophilic drug is a cannabinoid or vitamin E (or vitamin E derivative) or curcumin (or curcumin analogue or derivative). Preferably, the vitamin E derivative is selected from tocopherol acetate, tocopherol glucoside, tocopherol phosphate, tocopherol nicotinate and/or tocopherol succinate. Preferably, the vitamin E derivative is tocopherol succinate. This composition may have any of the appropriate features described below in the sixth aspect (process of making a solid composition comprising lipophilic drug and cyclodextrin).

The person of skill in the art will appreciate these classes of compounds share significant structural similarities (the cannabinoids, tocopherols and curcumin, and like compounds).

Suitably the lipophilic drug additionally has a size that is smaller than the size of the cavity of the CD, such as beta-cyclodextrin. Preferably, the lipophilic drug is between 100 and 700 g/mol. More preferably, the lipophilic drug is between 200 and 600 g/mol. Additionally, such a lipophilic drug optionally has a logP between 3 and 9, preferably between 3 and 8 and more preferably between 3 and 7 or between 4 and 9, preferably between 4 and 8 and more preferably between 4 and 7, or between 5 and 9, preferably between 5 and 8, more preferably between 5 to 7.

The structure of Vitamin E is shown below (with minor variation on the ring substitution providing for different tocopherol forms).

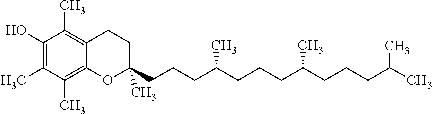 [SMILES: Cc1c(C)c2c(c(C)c1O)CC[C@@](C)(CCC[C@H](C)CCC[C@H](C)CCCC(C)C)O2]

The structure of curcumin is shown below and the skilled addressee will appreciate that keto-enol forms are present.

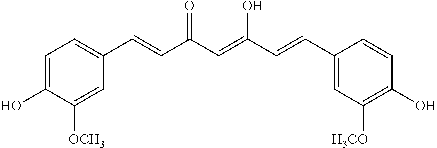 [SMILES: COc1cc(/C=C/C(=O)/C=C(O)/C=C/c2ccc(O)c(OC)c2)ccc1O]

The structural similarities between these compounds and the cannabinoids (discussed further below) is apparent.

Without wishing to be so limited, cannabinoids represent one class or group of lipophilic drugs which are well suited to use with the present invention. Cannabinoids are compounds found in the cannabis plant or synthetic compounds that can interact with the endocannabinoid system. As used herein, the term “cannabinoid” includes lipophilic compounds found in cannabis. At least 113 distinct cannabinoids have been isolated from cannabis.

As used herein, “cannabinoid” includes classical and non-classical cannabinoids. Classical cannabinoids are structurally related to THC. Nonclassical cannabinoids (cannabimimetics) include aminoalkylindoles, 1,5-diarylpyrazoles, quinolines, and arylsulfonamides as well as eicosanoids related to endocannabinoids.

In one embodiment, the cannabinoid is selected from the group consisting of cannabidiol (CBD), cannabinol (CBN), cannabigerol (CBG) and THC. THC may include delta-9-tetrahydrocannabinol and derivatives of this such as THCA-A, THCA-B, THCV and the like. In embodiments of the disclosure, the cannabinoid is preferably selected from CBD, CBG and delta-9-tetrahydrocannabinol. Most preferably, the cannabinoid is CBD and/or delta-9-tetrahydrocannabinol.

In another embodiment, the cannabinoid is cannabinol (CBN) or a tetrahydrocannabinol (THC), including delta-9-tetrahydrocannabinol.

While the cannabinoid family is large and encompasses a degree of structural diversity, it will be appreciated by a person of skill in the art that there are also significant common structural features. For example, the structures of some of the most important cannabinoids is shown below. The significant common structural features, including but not limited to a central phenolic ring with carbon chain substitution, are apparent.

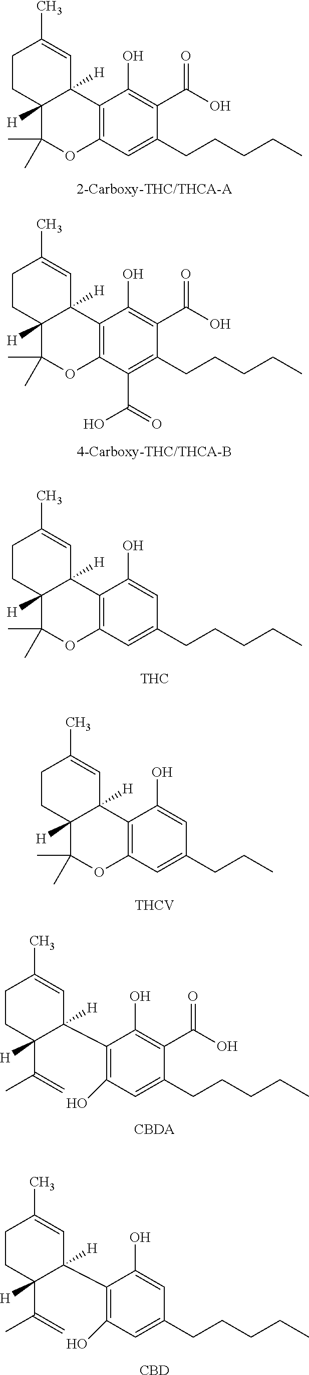

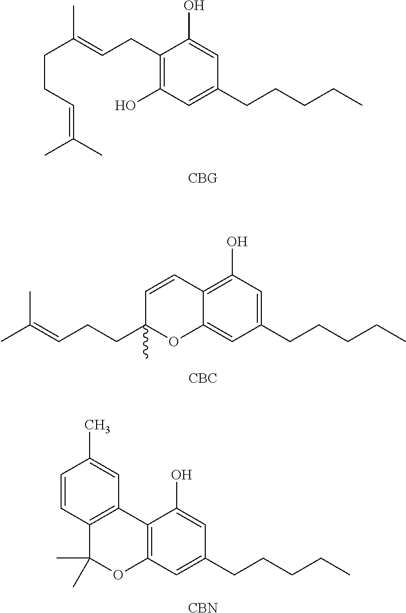

The examples shown herein make it clear that the size and lipophilicity of cannabinoids makes them particularly suitable for complexation with a CD using the process of the present disclosure.

Therefore, in embodiments in which the lipophilic drug is a cannabinoid, the cannabinoid may include the following Formula I structure:

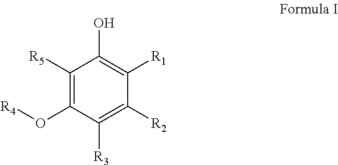

wherein,
R₁ and R₃ may be selected from hydrogen and carboxy;
R₂ is C₂-C₈ alkyl;
R₄ may be selected from hydrogen or C₂-C₈ alkyl;
R₅ may be selected from C₂-C₁₂ alkyl or alkenyl and a C₆ carbocycle; or
R₄ and R₅ may join to form one or two ring structures,
wherein each of the aforementioned groups, as appropriate, may be substituted or unsubstituted.

In embodiments, R₂ is C₃-C₅ alkyl, inclusive of n-propyl, n-butyl, and n-pentyl.

In embodiments, R₅ may be C₈-C₁₂ alkenyl, unsubstituted or substituted with methyl, ethyl or propyl, or substituted cyclohexenyl.

In embodiments in which R₄ and R₅ join to form one or two ring structures, they may form a fused bicyclic system. The bicyclic system is preferably as shown for THC and CBN in the above structures.

As used herein, “molar ratio” refers to the proportion of the number of moles of the compounds forming the complex.

According to the present invention, the molar ratio between the lipophilic drug, such as a cannabinoid, and the cyclodextrin is about 1:2. It is a benefit of the present disclosure that the molar ratio provided (i.e. 1:2, lipophilic drug:CD) will be obtained in high purity. That is, a high purity composition of 1:2 combined complexation will be provided.

As used herein, the combined complexation wherein the molar ratio between the lipophilic drug, such as a cannabinoid, and the cyclodextrin is about 1:2 refers to a complex wherein there are two separate molecules of cyclodextrin complexed with one molecule of cannabinoid. As such, the cyclodextrin is a non-bridged cyclodextrin (i.e. the cyclodextrin is not a bridged cyclodextrin). “Bridged cyclodextrin” includes cyclodextrin dimers wherein two molecules of cyclodextrin are covalently linked by a linker.

In embodiments of the first aspect, at least 80% by weight of the lipophilic drug, such as a cannabinoid, and the cyclodextrin in the dry solid composition are in the combined complexation at a molar ratio of about 1:2.

In more particular embodiments, at least 85% by weight of the lipophilic drug, such as a cannabinoid, and the cyclodextrin in the composition are in the combined complexation at a molar ratio of about 1:2, such as at least 90% by weight, preferably at least 95% by weight, more preferably at least 98% by weight, such as at least 99% by weight. In certain embodiments, substantially all of the lipophilic drug, such as a cannabinoid, and the cyclodextrin in the dry solid composition are in the combined complexation at a molar ratio of about 1:2.

The weight % is in respect of all lipophilic drug, such as a cannabinoid, and cyclodextrin present in the dry solid composition (i.e. in combined complexation at both 1:2 and 1:1 ratios and any uncomplexed lipophilic drug, such as cannabinoid, and cyclodextrin).

In other words, the combined complexation wherein the lipophilic drug, such as a cannabinoid, and the cyclodextrin are at a molar ratio of about 1:2 represents at least 80% by weight (such as at least 90% or 95% or 98% or 99% or even substantially all) with respect to the total lipophilic drug, such as cannabinoid, and cyclodextrin present in the dry solid composition.

“Percentage by weight” or “% by weight”, refer to the proportion of a particular substance within a mixture, as measured by weight or mass.

In embodiments of the first aspect, at least 80 mol % (such as at least 85 mol %, preferably at least 95 mol %, more preferably at least 98 mol %, such as at least 99 mol %) of the lipophilic drug, such as a cannabinoid, and the cyclodextrin in the composition are in the combined complexation at a molar ratio of about 1:2.

The term “mol %” refers to the proportion of a particular substance within a mixture, as measured by moles.

The term “about” when qualifying a number or value, is used herein to refer to values that lie within +10% of the value specified.

In embodiments, the solid composition of the first aspect may comprise a combined complexation of a lipophilic drug, such as a cannabinoid, having a logP between about 3 to about 8, and an unmodified alpha-, beta- or gamma-cyclodextrin;
wherein at least 80% by weight (such as at least 90% or 95% or 98% or 99% or even substantially all) of the total lipophilic drug, such as a cannabinoid, and alpha-, beta- or gamma-cyclodextrin present in the dry solid composition is in about a 1:2 molar ratio. Preferably, the cyclodextrin is unmodified beta-cyclodextrin. Preferably, the lipophilic drug has a size between 100 and 700 g/mol, more preferably between 200 and 600 g/mol or between 250 and 550 g/mol.

In embodiments, the solid composition of the first aspect consists of a combined complexation of a lipophilic drug, such as a cannabinoid, having a logP between about 3 to about 8, and an unmodified alpha-, beta- or gamma-cyclodextrin;
wherein at least 80% by weight (such as at least 90% or 95% or 98% or 99% or even substantially all) of the total lipophilic drug, such as a cannabinoid, and alpha-, beta- or gamma-cyclodextrin present in the dry solid composition is in about a 1:2 molar ratio. Preferably, the cyclodextrin is unmodified beta-cyclodextrin. Preferably, the lipophilic drug has a size between 100 and 700 g/mol, more preferably between 200 and 600 g/mol or between 250 and 550 g/mol.

It is an advantage of the presently disclosed process that a high degree of control is provided over the complexation process and so, while other approaches may provide a random mixture of various molar ratios of lipophilic compound to CD along with uncomplexed lipophilic compound and uncomplexed CD, the present approach allows for the desired combined complexation product at a high degree of purity without the need for subsequent complex additional purification steps.

In one embodiment, the solid composition of the first aspect consists of a combined complexation of a lipophilic drug, such as a cannabinoid, having a logP between about 3 to about 8, or between about 3 to about 7, and an unmodified beta-cyclodextrin;
wherein at least 90% or 95% or 98% or 99% by weight, or even substantially all, of the total lipophilic drug, such as a cannabinoid, and beta-cyclodextrin present in the dry solid composition is in the combined complexation in about a 1:2 molar ratio, optionally wherein the lipophilic drug is selected from the group consisting of CBD, delta-9-THC, CBG, CBN, vitamin E (or a vitamin E derivative) and curcumin (or a curcumin analogue or derivative). Preferably, the lipophilic drug has a size between 100 and 700 g/mol, more preferably between 200 and 600 g/mol or between 250 and 550 g/mol.

The solid compositions of the invention may be formulated with a pharmaceutically acceptable carrier. By “pharmaceutically acceptable carrier” is meant any diluent or excipient, such as fillers or binders, that is compatible with the other ingredients of the compositions, and which is not deleterious to the recipient. The pharmaceutically acceptable carrier can be selected on the basis of the desired route of administration, in accordance with standard pharmaceutical practices.

The compositions of the first aspect of the invention can conveniently be collected as a solid composition, such as a precipitate, and easily dried due to the lack of any requirement for large volumes of water or other solvent. They can then be measured out precisely and formulated for any desirable route of administration. Commercially available cannabinoid formulations rely upon liquid formulations using solvents designed to maximise dissolution of the lipophilic cannabinoid. The present solid compositions, due to the improved solubility profile they provide, may be formulated in aqueous formulations and so open up pharmaceutical grade formulations for injection as well as direct administration of the solid composition in a solid form.

The compositions of the first aspect of the invention may be formulated to be administered orally, intravenously, subcutaneously, dermally, sublingually, topically, ophthalmically (e.g. via eye drops), by any other parenteral route, in a pharmaceutically acceptable dosage form. The resulting pharmaceutical compositions will include compositions in the form of tablets, capsules or elixirs for oral administration, sterile solutions or suspensions for parenteral or intramuscular administration, and the like. Alternatively, particularly where compositions of the first aspect of the invention act locally, such compositions may be formulated for topical administration.

In an embodiment, the compositions of the first aspect are formulated in liquid forms suitable to be taken orally or by injection, or forms suitable for topical administration. These liquid forms may also contain stabilizing ingredients and/or buffering ingredients and are dispensed into unit doses in the form of ampoules or vials. The liquid forms (i.e. solutions) may also be prepared as a dry preparation to be reconstituted with a suitable solvent extemporaneously before use.

In some embodiments, the compositions of the first aspect are formulated as eye drops.

In some embodiments, the compositions of the first aspect are formulated as injections, such as injections suitable for administration to the face.

In an embodiment, the compositions of the first aspect are formulated such that they are suitable for oral administration, for example as a tablet, capsule, pellet, gum, or powder, preferably a tablet or capsule. By “oral composition” and “oral administration” it is meant the composition is suitable for swallowing.

Tablets, capsules, pellets, and gums may be prepared with binding agents, for example, acacia, syrup, gelatin, sorbitol, tragacanth, celluloses or polyvinylpyrrolidone; fillers, such as lactose, sucrose, corn starch, calcium phosphate, sorbitol, or glycine; lubricants, such as magnesium stearate, talc, polyethylene glycol, or silica; and surfactants, such as sodium lauryl sulfate.

Compositions of the first aspect may also be formulated as liquid compositions for oral administration. For example, in the form of syrups or suspensions, e.g. solutions or suspensions, containing a composition of the first aspect of the invention and the remainder of the formulation consisting of sugar or sugar alcohols, and a mixture of ethanol, water, glycerol, propylene glycol and polyethylene glycol. If desired, such liquid preparations may contain colouring agents, flavouring agents, saccharine and carboxymethyl cellulose or other thickening agent. Liquid preparations for oral administration may also be prepared in the form of a dry powder to be reconstituted with a suitable solvent prior to use.

In some embodiments, the compositions of the first aspect are formulated as mouth drops.

The compositions may also be formulated with an agent which reduces degradation of the substance by processes other than the normal metabolism of the patient, such as anti-bacterial agents, or inhibitors of protease enzymes which might be the present in the patient or in commensal or parasite organisms living on or within the patient, and which are capable of degrading the compound.

The formulated composition may be administered once a day, twice a day or three times a day.

It will be appreciated that the therapeutic uses of the solid composition of the first aspect are not particularly limited. Indeed, the disclosure of the present invention provides a platform approach by which a wide range of lipophilic drugs can be complexed with CDs. The complexed lipophilic drug will then be released in vivo to assert its therapeutic effect in the usual way without interference from the CD. The therapeutic use will therefore depend entirely on the particular lipophilic drug which has been complexed into the solid composition of the first aspect and will simply be in keeping with its already known and validated efficacy. The present approach simply allows for improved formulation and delivery of the known drug, amongst other advantages.

An effective dose will be apparent to one skilled in the art, and is dependent on a number of factors including age, sex, weight, and disease, which the medical practitioner will be capable of determining.

In an embodiment of the invention, the formulated composition is administered at least once a day. Preferably it is administered as a single daily dose. Preferably the single daily dose is 5 mg to 1000 mg, more preferably 10 mg to 500 mg, even more preferably 15 mg to 200 mg, such as 20 to 150 mg. Exemplary doses are 5 mg, 10 mg, 20 mg, 50 mg, 100 mg, 200 mg, 500 mg, 1000 mg.

In an embodiment of the invention, the formulated composition comprises between 10 mg and 1000 mg of lipophilic drug, such as a cannabinoid, such as 20 mg and 900 mg, 30 mg and 800 mg, 40 mg and 700 mg, 50 mg and 600 mg, 60 mg and 500 mg, 70 mg and 400 mg, 80 mg and 300 mg, and 90 mg and 200 mg. Preferably, the composition comprises between 200 mg to 750 mg lipophilic drug, such as a cannabinoid, more preferably 300 mg to 600 mg of lipophilic drug, such as a cannabinoid.

Suitably the formulated composition comprises more than 10 mg lipophilic drug, such as a cannabinoid, such as more than 20 mg, 50 mg, 100 mg, 200 mg, or 250 mg. Suitably, the formulated composition comprises less than 1000 mg of lipophilic drug, such as a cannabinoid, such as less than 800 mg, 700 mg, 600 mg, 500 mg, 400 mg, or 300 mg. Any of the aforementioned lower or upper limits may be combined with each other, and are herein disclosed.

In an embodiment of the invention, the formulated composition is administered at least twice a day. Preferably each dose is 2 mg to 500 mg, more preferably 3 mg to 300 mg, even more preferably 5 mg to 200 mg. Exemplary doses are 5 mg, 10 mg, 20 mg, 50 mg, or 100 mg.

In an embodiment of the invention, the formulated composition is administered at least three times a day. Preferably, each dose is 2 mg to 350 mg, more preferably 3 mg to 200 mg, even more preferably 5 mg to 100 mg. Exemplary doses are 5 mg, 10 mg, 20 mg, 50 mg, or 100 mg.

Preferably, the dosage regime is such that the total daily dosage of the lipophilic drug, such as a cannabinoid, does not exceed 1000 mg.

A second aspect of the invention is the solid composition according to the first aspect for use in the treatment of a disease, disorder or condition.

In an embodiment of the invention, the compositions are administered in an effective amount to treat a disease or disorder associated with the endocannabinoid system.

In some embodiments, the solid composition of the first aspect is for use as a medicament. In alternative embodiments, there is provided the use of a solid composition of the first aspect in the manufacturing of a medicament. In alternative embodiments, there is provided a method of treatment comprising administering to the patient a composition of the first aspect.

Suitably the solid composition is for use in the treatment of a disease or disorder associated with the endocannabinoid system.

“Patient” and “subject” are used interchangeably and refer to the subject that is to be administered the composition of the first aspect of the invention. Preferably, the subject is a human.

The disease or disorder associated with the endocannabinoid system is selected from the group consisting of anxiety, insomnia, epilepsy, neuropathic pain, opioid addiction, PTSD, IBD, stroke, acne, dermatitis, psoriasis, ADHD, SARS, inflammatory diseases, and chronic pain.

In a preferred embodiment, the disease or disorder associated with the endocannabinoid system is selected from the group consisting of anxiety and insomnia.

In an embodiment, the cannabinoid is the only active agent in the composition. By “only active agent” it is meant that the composition does not contain other components which may be used in the treatment of the disease or disorder associated with the endocannabinoid system.

In order to treat a disease or disorder associated with the endocannabinoid system, the solid composition of the first aspect of the invention is used in a chronic dosage regime i.e. chronic, long-term treatment. Suitably the regime lasts for at least one month, suitably at least two months, such as at least three months.

According to the third aspect of the invention, there is provided a method of treatment of a disease, disorder or condition comprising administering to a subject the solid composition of the first aspect of the invention.

In embodiments of the third aspect, the disease, disorder or condition will be determined by selection of the lipophilic drug based upon its known efficacy.

In embodiments of the third aspect, the disease, disorder or condition is one associated with the endocannabinoid system.

Embodiments of the third aspect of the invention may have any of the features described for the first and second aspects of the invention.

It will be appreciated that while the solid composition of the first aspect is administered to the subject, this does not require the composition still be in solid form. That is, the solid composition of the first aspect will be formulated in one form or another to be suitable for the desired route of administration. This may require the solid composition of the first aspect be formulated into a solution prior to administration. This, and similar approaches, are encompassed within the step of “administering to a subject the solid composition of the first aspect of the invention”.

According to the fourth aspect of the invention, there is provided the use of the solid composition of the first aspect (including the alternative first aspect) of the invention in the manufacture of a medicament for the treatment of a disease, disorder or condition.

In embodiments of the fourth aspect, the disease, disorder or condition will be determined by selection of the lipophilic drug based upon its known efficacy.

In embodiments of the fourth aspect, the disease, disorder or condition is one associated with the endocannabinoid system.

Embodiments of the fourth aspect of the invention may have any of the features described for the first to third aspects of the invention. This includes that the final form of the medicament may be, but is not limited to, a solid form.

According to the fifth aspect of the invention, there is provided the non-therapeutic use of the solid composition of the first aspect of the invention.

Suitably, the non-therapeutic use may be a use as a cosmetic, supplement, nutraceutical or for nutrition or in a beverage.

In some embodiments, the use is as a cosmetic, such as anti-acne, anti-aging, eczema, dry skin, sunburn, psoriasis or fungal infections.

In some embodiments, the use is as a supplement. Supplements are known as substances used to add nutrients to a person's diet or to lower a person's risk of health problems.

As used herein, a “nutraceutical” is a product derived from food sources that is purported to provide health benefits in addition to the basic nutritional value found in foods. A beverage is known as a liquid intended for human consumption. The composition of the invention may be mixed with one or more liquids to make a beverage.

Preferably, the non-therapeutic use is as a nutraceutical or for nutrition. Embodiments of the fifth aspect of the invention may have any of the appropriate features described for the first to third aspects of the invention.

According to the sixth aspect of the invention, there is provided a process of making a solid composition comprising a lipophilic drug and a cyclodextrin, wherein:
the lipophilic drug and the cyclodextrin are in the form of a combined complexation at a molar ratio of about 1:2,
wherein the process comprises the steps of:
a. providing the lipophilic drug;
b. providing the cyclodextrin;
c. combining the lipophilic drug and the cyclodextrin in the presence of water to form a composition;
d. mixing the composition until the combined complexation of lipophilic drug and cyclodextrin precipitates;
e. collecting the precipitate of step d. to provide the solid composition comprising the lipophilic drug and the cyclodextrin in the form of a combined complexation.

In embodiments, the lipophilic drug and cyclodextrin are in a saturated solution in step c. Advantageously, the saturation drives the complete complexation of the lipophilic drug and cyclodextrin.

In embodiments, the cyclodextrin is in at least slight excess over the lipophilic drug. This allows for substantially all of the lipophilic drug to be used up and placed into the combined complexation and provides for high purity product. If the excess cyclodextrin is present in a slight excess then when all of the lipophilic drug has been used up the cyclodextrin may be in solution and the combined complexation can be collected as the only solid product. If the cyclodextrin is in such excess that some solid remains then it is a simple step of purification to separate that from the combined complexation based on, for example, their respective solubilities.

Prior art approaches (for example, Mannila, J. et al, J. Pharm Sci, vol. 96, No. 2, 2007) rely upon a “precipitation complexation” reaction originally described by Higuchi et al (1965 Advances in analytical chemistry instrumentation, Wiley and sons, pp 117-212). However, this approach relies on the addition of an excess of the active compound to be complexed, into highly dilute solutions of the cyclodextrin. The reaction is then left to complex. The present inventor has found that this approach can take extremely long periods of time to proceed and so the prior approaches which collect the material relatively quickly are inevitably collecting large quantities of the active compound which has precipitated out, potentially with some precipitated cyclodextrin and then some unknown degree of actual complexed material which in itself potentially represents different molar ratios of complex. When the active compound is measured in such products, the level found is simply a representation of all the compound present in the various free and complexed forms and so does not give a true representation of the level of complexation. Further, the precipitation method of Mannila does not provide for a consistent and reliable high purity 1:2 lipophilic drug to cyclodextrin complex and so the advantages described above for such a complex cannot be achieved.

The present approach relies on saturation of the lipophilic drug and cyclodextrin to drive the reaction both in reasonable time frames and to completion. Hence, a high purity and reproducible combined complexation is obtained. The maintenance of a saturated solution of both lipophilic drug and cyclodextrin also ensures the convenient and largely complete precipitation of the combined complexation product until the excess starting material(s) in the system are used up and pushed into dissolution. This approach and the benefits are demonstrated in the examples herein in which, for example, CBD and beta-cyclodextrin are complexed. While these materials have been used in the precipitation complexation approach and an improvement in solubility of the final material was observed (from around 0.2 micromoles for CBD to about 3 micromoles for the final mixed product), the present approach gave a combined complexation of CBD:CD with a water solubility in the 1.5 to 2.7 millimole range (measured at 35-40° C., i.e. body temperature, with even greater solubilities achieved at higher temperature). Such a substantial increase in solubility could only be observed if the precipitation complex of the prior art did not, in fact, have all CBD complexed with the CD and the present approach provided for a solid composition in which substantially all of the CBD is complexed in the desired CBD:CD 1:2 molar ratio).

Without wishing to be bound by theory, the process of the sixth aspect of the invention is efficient because the reaction equilibrium concentration of the desired dimer complex is greater than the solubility of that dimer complex product in the reaction media. Simply put, this means the combined complexation product is constantly precipitating during the reaction and the cyclodextrin and lipophilic drug, such as a cannabinoid, are constantly dissolving to replace the cyclodextrin and lipophilic drug, such as a cannabinoid, lost from the liquid phase due to precipitation. Additionally, this means the concentration of all species in the liquid phase are constant throughout until excess materials are exhausted. The dimer can therefore be consistently and reliably achieved based upon these saturation, solubility and concentration dynamics parameters that have not been previously appreciated in the art.

The process enables the formation of lipophilic drug: cyclodextrin inclusion complex, in a 1:2 molar ratio (i.e. dimer complex), as described in detail in the Examples section and shown in FIG. 3. As discussed, it is believed that for a 1:2 complex the lipophilic drug will remain complexed to one cyclodextrin molecule when the dimer dissociates (i.e. the 1:2 drug:CD dimer dissociates to one equivalent of 1:1 drug:CD monomer and one equivalent of the cyclodextrin), which enables its absorption into the bloodstream. Otherwise, if only the monomer were to be formed and administered initially, the monomer would dissociate directly into the free cyclodextrin and free lipophilic drug, the lipophilic drug would precipitate and be excreted and it would therefore not be absorbed. The 1:2 dimer is therefore particularly important for poorly water-soluble drugs which are to be delivered in vivo.

The novel process described herein surprisingly allows for formation of the described dimer complex with a high degree of control thereby pushing the formation to completion with predictability and reproducibility. This provides for a surprising improvement in the solubility of the lipophilic drug and/or its bioavailability when delivered to a subject. It has not been previously realised that control over these saturation/concentration and excess material parameters could provide such surprising benefits in achieving substantially complete complexation of the lipophilic drug. The benefits cannot be achieved with any approach, such as those of the prior art, which relies upon a dilute (i.e. non-saturated) solution of lipophilic drug/cyclodextrin during mixing.

The solid composition formed by the process may have improved stability in aqueous and acidic medium (e.g. during and after preparation, and in the human stomach).

Additionally, the dimer has lower aqueous solubility compared to the monomer (i.e. lipophilic drug complexed with cyclodextrin in 1:1 molar ratio) which facilitates the preparation process, as the dimer precipitates selectively out of the aqueous reaction mixture.

Additionally, the lipophilic drug (i.e. the guest molecule) may be less soluble than both the cyclodextrin and the formed monomer or dimer complex. The complexation of the lipophilic drug into the cyclodextrin will continue to drive the lipophilic drug into solution. The dimer has lower solubility than the free cyclodextrin.

Measurement of logP can be undertaken in a variety of ways that are well-known to the skilled person. The lipophilicity of the drug must be suitable for interacting with the hydrophobic interior of the cyclodextrin, in order for the drug:cyclodextrin complex to form. In one embodiment, the lipophilic drug has a logP between 1 and 10.

Suitably, the lipophilic drug has a logP of more than 1, such as more than, 2, 3, 4, or 5. Suitably, the lipophilic drug has a logP of less than 10, such as less than 9, 8, or 7. Any of the aforementioned lower or upper limits may be combined with each other, and are herein disclosed.

Preferably the logP of the lipophilic drug is greater than or equal to 3, such as between 3 and 9, preferably between 3 and 8 and more preferably between 3 and 7 or between 4 and 9, preferably between 4 and 8 and more preferably between 4 and 7, or between 5 and 9, preferably between 5 and 8, more preferably between 5 to 7.

The size of the lipophilic drug must be suitable for allowing the drug to fit into the cyclodextrin cavity. Suitably the lipophilic drug has a size that is smaller than the size of the cavity of beta-cyclodextrin.

Suitably, the lipophilic drug has a size of more than 100 g/mol, such as more than 150, 200, or 250 g/mol. Suitably, the lipophilic drug has a size of less than 700 g/mol such as less than 650, 600, 550, 500, 450, 400, or 300 g/mol. Any of the aforementioned lower or upper limits may be combined with each other, and are herein disclosed.

Preferably, the lipophilic drug is between 100 and 700 g/mol. More preferably, the lipophilic drug is between 200 and 600 g/mol, or between 300 and 600 g/mol, or between 300 and 550 g/mol, or between 250 and 550 g/mol.

The lipophilic drug of the sixth aspect may be as described in any one or more embodiments of the first aspect.

Suitably the dimer complex (lipophilic drug:CD₂) is less soluble than the monomer complex (lipophilic drug:CD), which helps said dimer to precipitate conveniently in the manufacturing process.

Suitably the dimer complex (lipophilic drug:CD₂) is less soluble than the cyclodextrin, which helps said complexation to precipitate conveniently in the manufacturing process.

Without wishing to be bound by theory, the water is believed to enable the complexation to occur, thus acting as a mediator or a catalyst. However, in the presence of large amounts of water (i.e. low concentration of the lipophilic drug and/or cyclodextrin), the reaction mixture is too diluted, and the reaction does not occur or is excessively slow or does not go to completion appropriately. This is the case with the precipitation complexation approach of the prior art. The fastest rate is obtained when the reactants, cyclodextrin and lipophilic drug are maintained at their saturating concentrations.

Suitably in step d, the mixing of the composition continues until a solid precipitate forms. In some instances this may take between 0 and 48 hours, such as between 0 and 24 hours, for example between 0 and 12 hours. Suitably, the mixing occurs for at least 2 hours, preferably at least 4 hours, preferably at least 6 hours, such as at least 8 hours. Suitably, the mixing occurs for no more than 24 hours, preferably no more than 20 hours, more preferably no more than 16 hours, such as no more than 12 hours. For the avoidance of doubt, any of these lower or upper limits may be used in combination.

It has been found by the inventor that the order of addition of the lipophilic drug and the cyclodextrin do not impact the reaction. i.e., the process works when the lipophilic drug is added first to the water followed by the cyclodextrin, and when the cyclodextrin is added first to the water followed by the lipophilic drug, and when they are added simultaneously.

A “saturated solution” contains the maximum concentration of a solute dissolved in the solvent. As used herein, a “saturated solution” includes solutions comprising excess undissolved solute. Saturation may be measured by any technique known in the art.

For instance, saturation may be determined visually or calculated using the solubility values of the solutes.

Suitably the quantity of the lipophilic drug and the quantity of the cyclodextrin both exceed their relative solubilities in the water or water mixture. Suitably, the concentration of the lipophilic drug in the composition of step c is at saturation. Suitably, the concentration of cyclodextrin in the composition of step c is at saturation.

The saturation of the solutions may be maintained by having excess lipophilic drug and/or cyclodextrin in the reaction vessel, so that as the complexation progresses and the complex precipitates, more of the excess lipophilic drug and/or cyclodextrin dissolve. Preferably, the cyclodextrin is in excess in the composition of steps c. and d.

In one embodiment, the composition of step c is a solution with substantially all of the lipophilic drug dissolved in said solution and/or substantially all of the cyclodextrin dissolved in said solution.

In one preferred embodiment, the composition of step c comprises excess undissolved lipophilic drug and/or cyclodextrin. The excess material will dissolve as combined complexation product precipitates and so will continue to drive further complexation. A person of skill in the art will appreciate absolute amounts can be calculated based upon the desired amount of product relying on the present disclosure.

In one embodiment, the composition of step c comprises between 0.01% to 20%, 0.05% to 15%, 0.1% to 10%, 0.2% to 5%, 0.25% to 3%, or 0.3% to 2% of undissolved lipophilic drug and/or 2% and 90%, such as 2% to 80%, 3% to 70%, 5% to 60%, 10% to 60%, 15% to 50%, or 20% to 40% of undissolved cyclodextrin by weight of the composition. Suitably the composition of step c comprises between 2% and 90%, such as 2% to 80%, 3% to 70%, 5% to 60%, 10% to 60%, 15% to 50%, or 20% to 40% undissolved lipophilic drug and cyclodextrin by weight of the composition. Preferably, the composition of step c comprises between 2% to 60% undissolved lipophilic drug and/or cyclodextrin by weight of the composition.

An additional advantage of the process described herein is that it is not necessary to dissolve the cyclodextrin in a large amount of water to make the complex and hence the subsequent drying time as well as cost are greatly reduced compared to other methods. The cyclodextrin simply needs to be in a saturated solution in the water, whatever the volume of the system. As discussed above, excess undissolved cyclodextrin may well be desirable to maintain saturation as the cyclodextrin in the saturated solution combines with the lipophilic drug and precipitates from the liquid phase.

The cyclodextrin of the sixth aspect may be as described for any one or more embodiments of the first aspect. In a preferred embodiment of the sixth aspect, the cyclodextrin is native alpha-, beta-, or gamma cyclodextrin wherein ‘native’ means the cyclodextrin is unmodified. Preferably, the cyclodextrin of the sixth aspect is native beta-cyclodextrin.

A further advantage of the process of the sixth aspect of the invention is that the lipophilic drug and the cyclodextrin do not need to be micronised or nanoised prior to dissolving in the solvent (here water), thus allowing for a more simple and efficient process compared to methods that require the substrate to be micronised or nanoised.

Another method of monitoring the concentrations of the lipophilic drug and/or CD is by measuring the refractive index (RI) (which is commonly used and known in the field). Suitably, in step d, CD is constantly added to the starting CD:CBD mix from step c until the RI for CD is no longer able to be detected, which means that all the CD molecules in the solution have been complexed. There are other methods, including HPLC, which can also be used to indicate progression of the complexation.

The use of an RI measurement (such as by using an RI meter) in conjunction with the molar ratio of the cannabinoid to cyclodextrin allows for determining when the desired 1:2 combined complexation has formed and the reaction has completed. The RI of the reaction mixture when diluted to be within the solubility range of the total amount of cyclodextrin within the reaction will fall as the reaction proceeds. When the RI stops falling and becomes stable the reaction is complete as no further cyclodextrin is being complexed. As such, step d., may comprise taking one or more RI measurements. Advantageously, this allows for consistency between batches of material, as by using this method, each resulting batch has the same amount of the dimer, as opposed to batches containing varying amounts of the dimer and monomers.

In preferred embodiments, in step d., the mixing is continued until the cyclodextrin concentration is no longer at saturation. This indicates that substantially no cyclodextrin in the solid phase remains uncomplexed in the composition (i.e. the mixing is continued until substantially all cyclodextrin is contained in the solid complex precipitate). Achieving such control of the reactivity is advantageous for providing a highly efficient process and consistency between batches of material.

Suitably, in step c, the water is over 25° C., such as over 30° C., 35° C., 40° C., 45° C., 65° C., 75° C., 100° C. Preferably, the water is over 35° C., more preferably over 45° C. Suitably the water is about 50° C. Preferably, the water is below 80° C., more preferably below 70° C.

While heating is not essential it can provide benefits in speeding up the complexation and/or providing a high purity complexed product.

Without being bound by theory, some cyclodextrins result in a low yield of subsequent dimer complex when step c., occurs only with water due to the resulting complexes' higher solubility compared to the equilibrium concentration of the same. Therefore, in such instances, the yield of the complex can be increased by the addition of ethanol to the water, which changes the solubility and equilibrium concentration in step c., which causes the lipophilic drug to become more soluble and the cyclodextrin and the dimer complex less soluble, leading to a higher yield of the solid dimer product. As such, in some embodiments, in step c., the lipophilic drug and the cyclodextrin are mixed with a mixture of water and ethanol to form a composition. In particular embodiments, the mixture of water and ethanol comprises between 1 and 40 wt % ethanol, preferably between 5 wt % and 30 wt % ethanol, most preferably between 10 wt % and 30 wt %. Preferably in such embodiments, the cyclodextrin is alpha-cyclodextrin or gamma cyclodextrin.

Suitably, step e. may be undertaken using any standard technique e.g. filtration, decanting and removing the supernatant, centrifugation. Suitably the new solid precipitate is purified, for example, by drying off the excess water by known drying methods, such as freeze drying or air drying. It will be appreciated that the solid composition of the first aspect is effectively achieved as the combined complexation product precipitates. The invention is not particularly limited by the manner in which the precipitate solid composition product is collected as, in certain embodiments, it may even be transported, modified or used in its precipitated form from step d. The collecting may involve a direct collection of the solid precipitate or may, instead, involve removal of the water or aqueous phase in which the precipitate sits.

A further advantage of the process described herein is that the product in step e. is solid and can be purified by using standard techniques. Other known cyclodextrin complexes have to remain in liquid format as the water cannot be removed efficiently or cost effectively. The solid product of the present invention is advantageous compared to a liquid product because the solid is easier to transport and has increased stability by being in an inert powder form rather than a liquid format.

In some embodiments, in step d., the lipophilic drug and the cyclodextrin in the form of a combined complexation (i.e. the dimer) is formed in the solid phase in excess of 30% by weight solids to liquid. This is highly advantageous because the solid can be easily recovered by physical means such as filtration.

In specific embodiments, the process comprises the further step of drying and milling.

In one embodiment, the solid composition obtained by the process of the invention is suitable for oral administration.

In some embodiments, the process further comprises the step of:
f. forming a tablet, capsule, pellet, gum, or powder comprising the solid composition comprising the lipophilic drug and cyclodextrin extracted in step e, preferably forming a tablet or capsule.

Alternatively, the process further comprises the step of:
f. combining the solid composition comprising the lipophilic drug and cyclodextrin extracted in step e with appropriate carriers to form a gel, cream, or beverage. Such carriers include, but are not limited to, glycerol, glycerol monostearate, sodium citrate, citric acid, stearic acid, palmitic acid, beeswax substitute, acacia gum, sorbitol, malic acid, soy, lecithin, mannitol, flavours, phenylalanine, propylene glycol, polyethylene glycol, aqua, sodium, castor oil, methylparaben, or propylparaben.

In one embodiment of the sixth aspect, the lipophilic drug is a cannabinoid, Vitamin E or a vitamin E derivative, or Vitamin D derivative, a Vitamin A or Vitamin A derivative, a sterol or a sterol derivative, an antibiotic, curcumin or a curcumin analogue or derivative, a non-steroidal anti-inflammatory (NSAID), Artemesin or an Artemesin derivative. Preferably the lipophilic drug is a cannabinoid or Vitamin E, preferably a cannabinoid, more preferably a cannabinoid selected from the group consisting of CBD, CBN, CBG and THC.

Suitably the cannabinoid is cannabidiol (CBD), cannabinol (CBN) or tetrahydrocannabinol (THC), preferably CBD or THC. In a preferred embodiment, the cannabinoid is CBD.

Suitably, the vitamin E derivative may be selected from tocopherol acetate, tocopherol glucoside, tocopherol phosphate, tocopherol nicotinate and/or tocopherol succinate. Preferably, the vitamin E derivative is tocopherol succinate.

Suitably, the cyclodextrin is selected from the group consisting of a native alpha-, beta-, gamma cyclodextrin, and methyl beta cyclodextrin, preferably, the cyclodextrin is native alpha-, beta-, or gamma cyclodextrin. More preferably, the cyclodextrin is native beta-cyclodextrin.

In another embodiment, the cyclodextrin is hydroxypropylated-beta-cyclodextrin or sulfobutylated-beta-cyclodextrin.

For the avoidance of doubt, the process of the sixth aspect of the invention may have any of the features described above for the first to fifth aspects of the invention, including the lipophilic drug as described in any embodiment of the first aspect.

A seventh aspect of the invention is the solid composition obtainable (such as obtained) by the process of the sixth aspect of the invention.

The solid composition of the seventh aspect of the invention may have any of the features described above for the first to sixth aspects of the invention.

An eighth aspect of the invention is the solid composition of the first aspect when produced by the process of the sixth aspect.

The solid composition of the eighth aspect of the invention may have any of the features described above for the first to sixth aspects of the invention.

The invention will now be described by way of the following non-limiting examples.

The skilled person will understand that features which are optional can be used in different combinations to construct various different embodiments and examples of the invention not limited to those shown herein.

### EXAMPLES

### Example 1: Preparation of a CBD:Cyclodextrin Complex

A volume of 100 ml of water was heated to 50° C. and 40 g of beta cyclodextrin was added with stirring. The stirring was continued throughout the reaction. The quantity of solubilised beta cyclodextrin in the water was monitored until saturation was reached, as indicated by a stable cyclodextrin concentration. A quantity of 6 g of cannabidiol was added and again the quantity of solubilised beta cyclodextrin in the water was monitored. When the concentration of solubilised cyclodextrin was observed to fall to a minimum value and remain constant, more cyclodextrin was added gradually in 0.5 g aliquots until a soluble cyclodextrin concentration of 20 g/l was attained. The mixture was then allowed to cool to 20° C. before removal of the liquid phase by vacuum filtration. The filter cake was then dried to dryness over zeolite.

### Solubility Profile of Beta-CD in the Presence of CBD

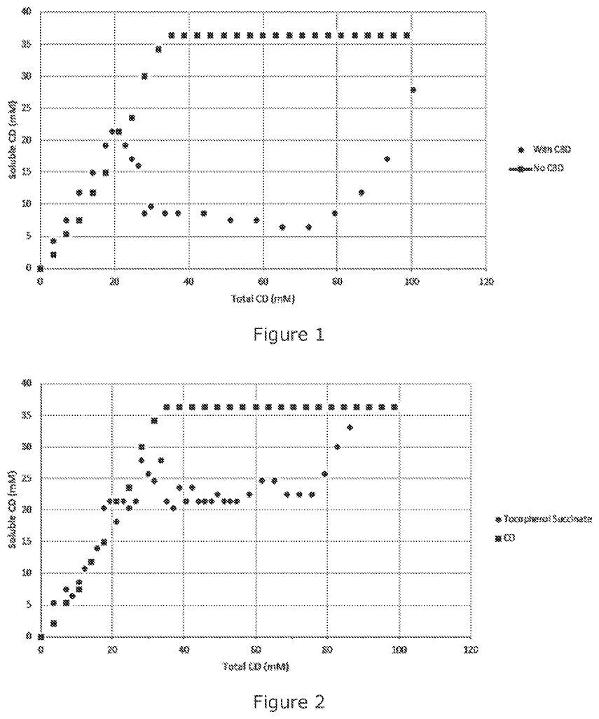

FIG. 1 shows that the solubility of beta cyclodextrin followed a characteristic shape when it was added in increasing quantities in the presence of a fixed amount of CBD. The black squares (CD on its own) show that the concentration measured by RI increased until it reached saturation and then remained unchanged as more cyclodextrin was added.

However, in the presence of CBD, the cyclodextrin followed the same solubility curve as before, but only up to a point. At about 20 mM, adding extra cyclodextrin actually caused the amount of soluble cyclodextrin to fall and it continued to fall until the soluble cyclodextrin concentration reached between 5 and 10 mM. After which adding more cyclodextrin made little difference to the soluble cyclodextrin concentration until about 80 mM was added, this coincided with a stoichiometric ratio of CBD to CD of 1:2, i.e. at this point all the CBD was complexed with cyclodextrin. Any more cyclodextrin added after this point could dissolve because there was no more CBD to bind to and the soluble concentration started to rise again.

### Example 2: Preparation of a Tocopherol Succinate (Vitamin E):Cyclodextrin Complex

A volume of 100 ml of water was heated to 50° C. and 40 g of beta cyclodextrin was added with stirring. The stirring was continued throughout the reaction. The quantity of solubilised beta cyclodextrin in the water was monitored until saturation was reached, as indicated by a stable cyclodextrin concentration. A quantity of 9.4 g of tocopherol succinate was added and again the quantity of solubilised beta cyclodextrin in the water was monitored. When the concentration of solubilised cyclodextrin was observed to fall to a minimum value and remain constant, more cyclodextrin was added gradually in 0.5 g aliquots until a soluble cyclodextrin concentration of 20 g/l was attained. The mixture was then allowed to cool to 20° C. before removal of the liquid phase by vacuum filtration. The filter cake was then dried to dryness over zeolite.

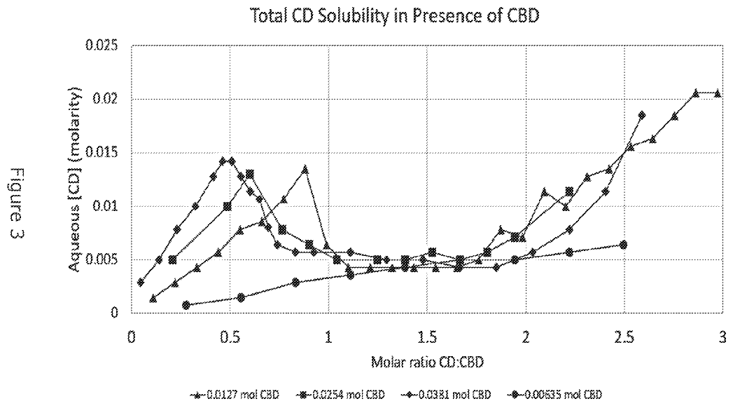

The solubility of CD in the presence of tocopherol succinate is shown in FIG. 2. The solubility of CD follows a similar profile as in Example 1 with CBD.

### Example 3: Preparation of a Curcumin:Cyclodextrin Complex

Curcumin (0.6 g) was added to 50 ml of water at 35° C. with stirring. Beta cyclodextrin powder was added to the mixture in successive 0.1 g quantities and allowed to equilibrate between additions. Equilibration was determined by a stable RI reading. The RI reading was recorded between subsequent additions. The soluble CD concentration was then plotted against the cumulative quantity of CD added.

The product (curcumin:cyclodextrin 1:2 dimer) was isolated in an analogous manner as Example 1.

### Example 4: Preparation of a CBG:Cyclodextrin Complex

32 g of beta cyclodextrin were added to a beaker and water was added to make a total volume of 400 ml. The mixture was heated to 55° C. with stirring and 3 g of CBG were added. Samples were taken at regular intervals and diluted 1 part in 5 with water at 20° C. before measuring the RI of the diluted sample.

The reaction was stopped when the RI of the diluted sample fell to 0.3 and became constant, indicating the reaction was complete. The reaction was allowed to cool to room temperature (approximately 20° C. before the solid phase was recovered by filtration, the filtrate liquid phase including unreacted dissolved beta cyclodextrin was discarded.

The dimer 1:2 CBG:CD product was obtained.

FIG. 7 shows the solubility of beta cyclodextrin in the presence of CBG.

### Example 5: Preparation a CBD:Alpha-CD Dimer Complex and a CBD:Gamma-CD Dimer Complex

The complexation reaction requires the reaction equilibrium concentrations of the dimer complexes formed to be greater than the solubility of those complexation products in the reaction media in order to achieve high yields of the dimer complex. When the reaction media is water, those conditions could not be met when using either alpha or gamma cyclodextrin to complex with CBD, and thus the yield of the reaction was low.

A successful complexation reaction is indicated by a fall in the refractive index on completion.

The following experiment investigated replacing water as the reaction media with diluted ethanol solution (i.e. a mixture of water and ethanol). The presence of ethanol in the aqueous reaction media both increases the solubility of CBD and decreases the solubility of cyclodextrins, and the CBD/cyclodextrin complexes.

To test whether using an aqueous ethanol solution as the reaction media, either alpha or gamma cyclodextrin were added to an aqueous ethanol solution in the presence of a quantity of CBD. A successful reaction would be indicated by a reduction in the refractive index following overnight incubation at 25° C.

### Results

| | | RI | RI | Successful |
|---|---|---|---|---|
| Cyclodextrin | Ethanol | T = 0 | T = O/N | Reaction |
| Gamma cyclodextrin | 25% (w/w) | 17.6 | 10.1 | Yes |
| Alpha cyclodextrin | 16.5% (w/w) | 10.4 | 9.4 | Yes |

As used in the table above, “O/N” refers to overnight.

### Conclusion

Using aqueous ethanol as the reaction medium altered the relative solubility of the reaction species so that the relative solubilities of the cyclodextrin/CBD complexation products were lower than the equilibrium concentrations of those products. This allowed the reaction to proceed to completion successfully.

Both dimer products (i.e. CBD:alpha cyclodextrin and CBD:gamma cyclodexrin) were prepared in high yields (>90% complexation efficiency) successfully.

### Assessment of CBD:CD Complex Stoichiometry

The results of this experiment are shown in FIG. 3.

Beta-Cyclodextrin was added in aliquots to water at 50° C. with stirring containing either 0.00635 mol CBD, 0.0127 mol CBD, 0.0254 mol CBD or 0.0381 mol CBD. The concentration of cyclodextrin was measured after each addition. The results show that the soluble cyclodextrin concentration increases linearly until a concentration of approximately 0.014 M is reached. This indicates that little reaction between the CD and CBD occurs at CD concentrations below 0.014 M. Paradoxically, increasing the quantity of CD added beyond 0.014 mM causes the concentration of aqueous (dissolved) CD to decrease rather than increase and this trend continues until the molar ratio of CD and CBD are approximately equivalent. The concentration of aqueous CD remains constant with increasing additions of CD when the molar ratios of CBD and CD are between 1 and 2. This suggests that between these molar ratios an equilibrium exists between CBD:CD monomer, CBD:CD₂ dimer and CD in solution. Additions of CD to mixtures with a CD:CBD ratio greater than 2 results in the concentration of aqueous CD increasing once again, showing that the former equilibrium is no longer maintained and free CD exists in solution.

In conclusion, the CD/CBD complexation reaction requires a concentration of CD that must be above a minimum quantity. The reaction is sequential, forming a monomer (CBD:CD) and then a dimer (CBD:CD₂). The CBD:CD monomer, CBD:CD₂ dimer and CD form a stable equilibrium in solution.

### Assessment of Stability of Dimer CBD/CD Complex

A stability trial of a technical batch of a CBD:beta-CD dimer product encapsulated in 2-piece hard shell capsules (420 mg per capsule) was conducted.

The stability study was run over 3 months at 25° C./60% RH and 40° C./75% RH and analysed by HPLC-UV/MS at t=0, 1, 2 and 3 month time points.

At each time point, the capsules were removed from the humidity chambers and prepared as follows. Samples were prepared in 250 ml volumetric flasks. This volume was found, during initial investigation, to be sufficient to bring the CBD concentration in each capsule into the calibration range. The capsules were opened by separating the two sides of the casing and the contents carefully emptied into the flask. The insides of the casings were washed 3 times with 50:50 methanol/water solution and the washings poured into the flask to ensure any residual powder was captured also. A cloudy solution was consequently formed. Each sample solution was subsequently sonicated using a Fisherbrand sonicator at a frequency of 37 kHz and a power of 40% for 30 minutes. Following sonication, the resulting solutions were transparent, indicating that the CBD/CD complex was fully dissolved.

After sonication, the samples were left to cool to room temperature prior to being topped up to the mark with additional 50:50 Methanol/water. Flasks were inverted 10 times to ensure the sample solutions were sufficiently mixed and aliquots were then removed to fill HPLC vials. Two sets of HPLC vials were prepared for each sample; one for analysis and one to be kept in the freezer in the event that any re-runs were required. All sample analysis was carried out on the same day as the sample preparation and a new calibration was acquired with each set of samples run. The regression lines for each calibration (acquired from injections of the certified reference standard) were used to calculate the concentrations of CBD in the samples and thus the mass of CBD each capsule.

The samples were subsequently analysed using the HPLC-UV/MS method outlined in the table below.

| Software Acquisition | Chemstation | | |
|---|---|---|---|
| and processing | | | |
| Wavelength | 228 nm, 275 nm | | |
| Column (type/size) | Phenomenex, Kinetex ®, 1.7 μm C18 | | |
| | 100 Å, 30 × 2.1 mm | | |
| Oven Temperature | 40° C. | | |
| Flow Rate | 0.7 mL/min | | |
| Injection Volume | 10 μL | | |
| Mobile Phase A | Water + 0.1% Formic Acid | | |
| Mobile Phase B | Methanol/Acetonitrile (3/1) + | | |
| | 0.1% Formic Acid | | |
| | Time (min) | A % | B % |
| Gradient Program | Initial | 40 | 60 |
| | 0.5 | 40 | 60 |
| | 5.3 | 25 | 75 |
| | 7.1 | 25 | 75 |
| | 7.12 | 0 | 100 |
| | 7.7 | 0 | 100 |
| | 7.8 | 40 | 60 |
| | 9 | 40 | 60 |

| | |
|---|---|
| Acquisition Time | 9 min |
| Diluent | MeOH/H₂O (50:50, v/v) |

For each set of samples analysed a new calibration was acquired and the corresponding regression equations were used to calculate the concentration of CBD in the 250 ml sample solution and subsequently the mass of CBD in each capsule.

Throughout the study all calibrations showed good reproducibility and accuracy and exhibited R² values above 0.9998.

UV chromatograms and MS Total Ion Chromatograms (TIC) were examined and compared for the pure CBD reference standard injections and all the samples analysed. There were no additional peaks observed in the sample chromatograms that didn't already exist in the standard. This implies that no degradation products are present in the samples analysed from the each timepoint and the duration of the study.

### DSC Analysis of the CBD/CD Complex

Differential Scanning Calorimetry (DSC) was used to evaluate the physical state of CBD within the CBD:CD dimer product prepared in accordance with Example 1 above.

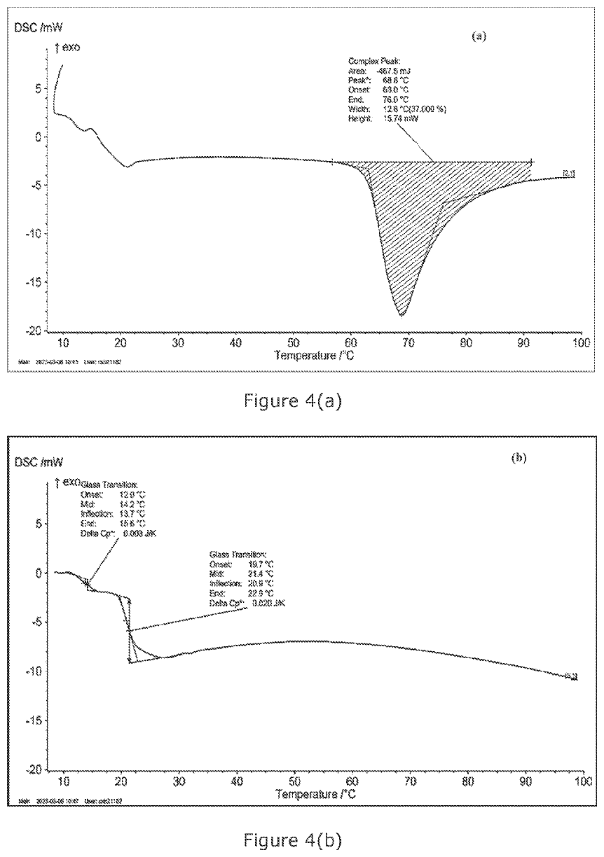

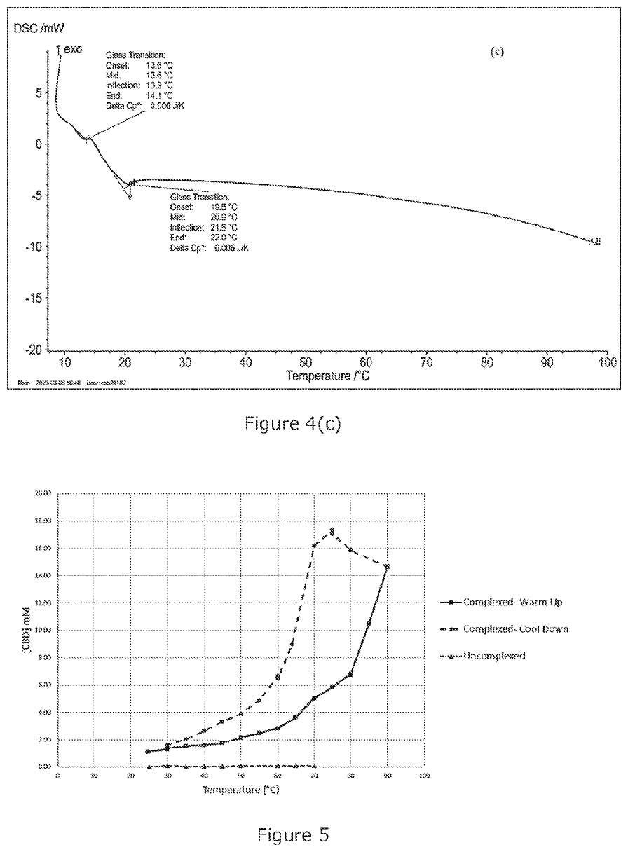

5 to 10 mg of powder were accurately weighed, crimped in an aluminium pan and then subjected to one heating ramp at a rate of 20° C./min ranging from 10° C. to 100° C. A heating rate of 20° C./min was found to be optimum for detecting crystallinity in formulations prepared by Koch et al. (Koch et al, 2020. Int. J. Pharm. 119812) and therefore the method was applied to this work. DSC measurements were collected using a Netzsch STA449 F1 Jupiter DSC/TGA with the Netzsch software-Proteus-61 (STA 449F1 on USBc1 coupled with the Autosampler for data collection and Proteus Analysis for processing). Blank correction was carried out with an empty aluminium pan pierced lid under the same measurement conditions prior to sample analysis. The DSC profiles of the CBD and CD raw materials and the CBD/CD complex are illustrated in FIG. 4(a) to (c), respectively.

FIG. 4 shows that the CBD/CD complex show the same glass transitions as the CD raw material. FIG. 4(c) shows no transition in the same region as the CBD material (68° C.) which suggests that the CBD is fully complexed.

### Assessment of the Solubility of the CD:CBD Dimer Complex (UV Analysis)

The concentration of the CD:CBD dimer complex was measured using UV analysis, in a solution containing 200 ml of water and 10 g of the dimer complex made in accordance with Example 1, whilst gradually increasing and then decreasing the temperature of the solution using a hot-plate stirrer.

The solubility profile is shown in FIG. 5. The solubility profile follows an unexpected pattern in that the solubility of complexed CBD during heating appears to be almost exponential. However, a different trend was observed on cooling. Instead, the CBD dimer seems to remain in solution for a period of time and the solubility decreases slowly as the temperature is reduced. This is unusual, as it would normally be expected that the solubility on cooling would simply be a reverse of the solubility on heating.

The CD:CBD dimer showed a solubility of 1.6 mM at around 37° C. (i.e. body temperature), increasing to 2.7 mM if the solution was allowed to heat beyond 60° C. before cooling back down to body temperature.

This result showed the significant increase in solubility of the dimer compared to the free CBD, whose solubility is known to be only around 0.2 μM. This also suggests that the dimer complex would have a much higher bioavailability compared to free CBD.

The maximum solubility of the CD:CBD dimer measured in this experiment was around 15 mM to 17 mM at 90° C. to 70° C. on the cooling cycle.

### Phase Solubility Diagrams

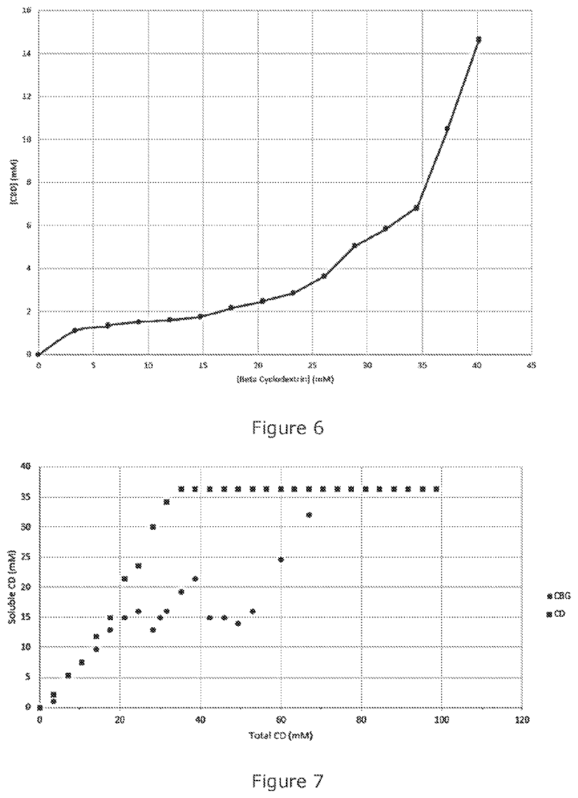

A phase solubility diagram of CBD/CD is shown in FIG. 6. The seminal literature for constructing a phase diagram is work by Takeru Higuchi and Kenneth Connors (Advances in analytical chemistry instrumentation, 1965, 4, 117-212). Using the Higuchi and Connors method it would be expected that CBD/CD would give apparent Type B complexation behaviour as described on page 148 of Higuchi et al.

Surprisingly, and in contradiction with this earlier literature, the phase solubility diagram in FIG. 6 shows Type A_{P} phase solubility behaviour. This means that for many lipophilic drugs (including CBD, Curcumin, Tocopherol, etc), the stoichiometry(s) of the complex cannot be estimated using a standard Higuchi & Connors phase solubility diagram. The potential increase in solubility estimated using a Higuchi and Connors phase solubility diagram is likely to be grossly underestimated and the amount of cyclodextrin needed to achieve solubility grossly overestimated.

### Assessment of Bioavailability

Two-piece hard shell capsules were prepared containing either 100 mg of cannabidiol or 417 mg of CBD:CD complex (50 mg CBD, equivalent) made in accordance with Example 1. Fasted subjects were administered either a single cannabidiol capsule (100 mg cannabidiol) or two cannabidiol/cyclodextrin capsules (total 100 mg cannabidiol equivalent) by mouth. The capsules were taken with a standard glass of water. Capillary blood was drawn one hour after ingestion and the quantity of cannabidiol and cannabidiol metabolites in blood analysed by a certified laboratory.

TABLE 1
Capillary blood 1 hour post ingestion

| | | Concentration | Reference range |
|---|---|---|---|
| | Agent | (ng/ml) | (ng/ml) |
| Plain CBD | CBD | 11.52 | 0.1-80 |
| | CBD-OH | 1.85 | 0.1-80 |
| | CBD-COOH | 13.14 | 0.5-400 |
| | Total | 26.77 | |
| CBD/CD Dimer of | CBD | 41.22 | 0.1-80 |
| the invention | CBD-OH | 15.69 | 0.1-80 |
| | CBD-COOH | 89.63 | 0.5-400 |
| | Total | 146.54 | |

The results show a four-fold increase in CBD concentration and a fivefold increase in total CBD and metabolites with cannabidiol/cyclodextrin dimer (complex) in capillary blood as compared with native cannabidiol.

### Conclusion

A complex between lipophilic drug (such as CBD or Vitamin E) and cyclodextrin in a 1:2 molar ratio has been shown to enable the preparation of a stable solid composition for oral administration. The resulting solid composition has increased bioavailability and can be used for oral administration without being excreted.

### Itemised Listing of Embodiments

1. A solid composition comprising a lipophilic drug and a cyclodextrin wherein the lipophilic drug and the cyclodextrin are in the form of a combined complexation and the lipophilic drug and the cyclodextrin in the combined complexation are at a molar ratio of about 1:2.
2. The solid composition of item 1 wherein at least 80% by weight of the lipophilic drug and the cyclodextrin in the solid composition are in the combined complexation at the molar ratio of about 1:2.
3. The solid composition of item 1 or item 2 wherein at least 90% by weight of the lipophilic drug and the cyclodextrin in the solid composition are in the combined complexation at the molar ratio of about 1:2.
4. The solid composition of any one of the preceding items wherein at least 95% by weight of the lipophilic drug and the cyclodextrin in the solid composition are in the combined complexation at the molar ratio of about 1:2.
5. The solid composition of any one of the preceding items wherein at least 97% by weight of the lipophilic drug and the cyclodextrin in the solid composition are in the combined complexation at the molar ratio of about 1:2.
6. The solid composition of any one of the preceding items wherein at least 98% by weight of the lipophilic drug and the cyclodextrin in the solid composition are in the combined complexation at the molar ratio of about 1:2.
7. The solid composition of any one of the preceding items wherein at least 99% by weight of the lipophilic drug and the cyclodextrin in the solid composition are in the combined complexation at the molar ratio of about 1:2.
8. The solid composition of any one of the preceding items wherein the lipophilic drug is the only therapeutically active agent in the solid composition.
9. The solid composition of any one of the preceding items wherein the solid composition comprises between 20 mg to 300 mg of the lipophilic drug, preferably between 40 mg to 200 mg of the lipophilic drug, more preferably between 50 mg to 150 mg of the lipophilic drug.
10. A formulation comprising the solid composition of any one of item 1 to item 9.
11. The formulation of item 11, wherein the solid composition is formulated into a tablet, capsule, pellet, gum, powder, gel, cream, paste, eye drop, pastille, ingestible solution, beverage, functional food or injectable formulation.
12. The formulation of item 11 or item 12, wherein the formulation comprises between 20 mg to 300 mg of lipophilic drug, preferably between 40 mg to 200 mg of lipophilic drug, more preferably between 50 mg to 150 mg of lipophilic drug.
13. The solid composition of any one of the preceding items for use in the treatment of a disease, disorder or condition.
14. A method of treatment of a disease, disorder or condition comprising administering to a subject the solid composition of any one of item 1 to item 9.
15. Use of the solid composition of any one of item 1 to item 9 in the manufacture of a medicament for the treatment of a disease, disorder or condition.
16. The solid composition for use of item 13, method of item 14 or use of item 15, wherein the disease, disorder or condition is one for which the lipophilic drug is known to have efficacy.
17. The solid composition for use, method or use of item 16, wherein the disease, disorder or condition is one associated with the endocannabinoid system, or one associated with a vitamin deficiency (such as a vitamin E deficiency) or one responsive to treatment with an antioxidant (such as curcumin).
18. The solid composition for use, method or use of item 16, wherein the disease, disorder or condition is selected from the group consisting of anxiety, insomnia, epilepsy, neuropathic pain, opioid addiction, PTSD, IBD, stroke, acne, dermatitis, wrinkles, psoriasis, ADHD, SARS, inflammatory diseases, and chronic pain.
19. A non-therapeutic use of the solid composition of any one of item 1 to item 9.
20. The non-therapeutic use of item 19, wherein the use is as part of a cosmetic formulation, a supplement, a nutraceutical or for nutrition, or a beverage.
21. A process of making a solid composition comprising a lipophilic drug and a cyclodextrin, wherein:
the lipophilic drug and the cyclodextrin are in the form of a combined complexation at a molar ratio of about 1:2,
   wherein the process comprises the steps of:
a. providing the lipophilic drug;
b. providing the cyclodextrin;
c. combining the lipophilic drug and the cyclodextrin in the presence of water to form a composition;
d. mixing the composition until the combined complexation of lipophilic drug and cyclodextrin precipitates;
e. collecting the precipitate of step d. to provide the solid composition comprising the lipophilic drug and the cyclodextrin in the form of a combined complexation.
   22. The process of item 21, wherein the combining and/or mixing steps are carried out at a temperature of between 25° C. to 70° C., such as between 30° C. to 70° C., or between 40° C. to 70° C., or between 45° C. to 70° C., or between 25° C. to 60° C., or between 30° C. to 60° C., or between 40° C. to 60° C., or between 45° C. to 60° C. 23. The process of item 21 or item 22, wherein the quantity of the lipophilic drug and/or the quantity of the cyclodextrin exceed their relative solubilities in the composition of step c.
   24. The process of any one of item 21 to item 23, wherein the concentration of the lipophilic drug in the composition of step c is at saturation.
   25. The process of any one of item 21 to item 24, wherein the concentration of the cyclodextrin in the composition of step c is at saturation.
   26. The process of any one of item 21 to item 25, wherein undissolved lipophilic drug and/or cyclodextrin are present in the combining step, so that as the complexation progresses and the solid composition precipitates, further lipophilic drug and/or cyclodextrin dissolve.
   27. The process of any one of item 21 to item 26, wherein the cyclodextrin is in molar excess over the lipophilic drug in the combining step and/or mixing step.
   28. The process of any one of item 21 to item 27, wherein in step d. the mixing is continued until substantially no uncomplexed (i.e. free) cyclodextrin and substantially no uncomplexed (i.e. free) lipophilic drug is present in the composition.
   29. The process of any one of item 21 to item 28, wherein an RI (refractive index) meter is used to indicate completion of the complexation.
   30. The process of any one of item 21 to item 29, wherein at least 80%, or at least 90%, or at least 95%, or at least 97%, or at least 98%, or at least 99%, by weight of the lipophilic drug and the cyclodextrin in the solid composition are in the combined complexation at the molar ratio of about 1:2.
   31. The process of any one of item 21 to item 30, wherein step d. is carried out in an aqueous alcohol (such as aqueous ethanolic) solution.
   32. A solid composition obtainable by the process of any one of item 21 to item 31.
   33. A solid composition when produced by the process of any one of item 21 to item 31.
   34. In any one of the preceding items, the cyclodextrin is a native (unmodified) alpha-, beta-, or gamma-cyclodextrin.
   35. In any one of the preceding items, the cyclodextrin is a native (unmodified) beta-cyclodextrin.
   36. In any one of the preceding items, the lipophilic drug has a size that is smaller than the size of the cavity of the cyclodextrin, such as the beta-cyclodextrin.
   37. In any one of the preceding items, the lipophilic drug is between 100 and 700 g/mol, more preferably between 200 and 600 g/mol, or between 250 and 550 g/mol.
   38. In any one of the preceding items, the lipophilic drug has a logP greater than or equal to 3, such as between 3 and 9, preferably between 3 and 8 and more preferably between 3 and 7.
   39. In any one of the preceding items, the lipophilic drug has at least one carbon chain which is at least 3-carbon atoms long.
   40. In any one of the preceding items, the lipophilic drug is a compound having a phenolic ring which may be substituted and/or fused with a further ring.
   41. In any one of the preceding items, the lipophilic drug is a compound having a phenolic ring which is substituted with an alkyl or alkenyl chain.
   42. In any one of the preceding items, the lipophilic drug is a compound having a phenolic ring which is substituted with an alkyl or alkenyl chain which is at least 3-carbons long.
   43. In any one of the preceding items, the lipophilic drug is a compound having a structure of Formula I:

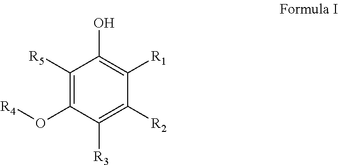

wherein,
R₁ and R₃ may be selected from hydrogen and carboxy;
R₂ is C₂-C₈ alkyl;
R₄ may be selected from hydrogen or C₂-C₈ alkyl;
R₅ may be selected from C₂-C₁₂ alkyl or alkenyl and a C₆ carbocycle; or
R₄ and R₅ may join to form one or two ring structures,
wherein each of the aforementioned groups, as appropriate, may be substituted or unsubstituted.
   44. The compound of item 43, wherein R₂ is C₃-C₅ alkyl, inclusive of n-propyl, n-butyl, and n-pentyl.
   45. The compound of item 43 or item 44, wherein R₅ is C₈-C₁₂ alkenyl, unsubstituted or substituted with methyl, ethyl or propyl, or substituted cyclohexenyl.
   46. The compound of any one of item 43 to item 45, wherein when R₄ and R₅ join to form one or two ring structures, they form a fused bicyclic system.
   47. In any one of the preceding items, the lipophilic drug is selected from the group consisting of a cannabinoid, Vitamin E or a vitamin E derivative, Vitamin D or a Vitamin D derivative, Vitamin A or a Vitamin A derivative, curcumin or a curcumin derivative, a sterol or a sterol derivative, an antibiotic, a non-steroidal anti-inflammatory (NSAID), and Artemesin or an Artemesin derivative.
   48. In any one of the preceding items, the lipophilic drug is a vitamin E derivative selected from tocopherol acetate, tocopherol glucoside, tocopherol phosphate, tocopherol nicotinate and/or tocopherol succinate, preferably, the vitamin E derivative is tocopherol succinate.
   49. In any one of the preceding items, the lipophilic drug is a cannabinoid selected from the group consisting of cannabidiol (CBD), cannabinol (CBN), cannabigerol (CBG) and delta-9-tetrahydrocannabinol (THC).

1. A process of making a solid composition comprising a lipophilic drug and a cyclodextrin, wherein:
the lipophilic drug and the cyclodextrin are in the form of a combined complexation at a molar ratio of about 1:2,
wherein the process comprises the steps of:
a. providing the lipophilic drug;
b. providing the cyclodextrin;
c. combining the lipophilic drug and the cyclodextrin in the presence of water to form a composition;
d. mixing the composition until the combined complexation of lipophilic drug and cyclodextrin precipitates;
e. collecting the precipitate of step d. to provide the solid composition comprising the lipophilic drug and the cyclodextrin in the form of a combined complexation,
wherein the quantity of the lipophilic drug and the quantity of the cyclodextrin exceed their relative solubilities in the composition of step c.

2. (canceled)

3. (canceled)

4. (canceled)

5. The process of claim 1, wherein undissolved lipophilic drug and/or cyclodextrin are present in the combining step c., so that as the complexation progresses and the solid composition precipitates, further lipophilic drug and/or cyclodextrin dissolve; and/or wherein the cyclodextrin is in molar excess over the lipophilic drug in the combining step c. and/or mixing step d.

6. (canceled)

7. The process of claim 1, wherein in step d. the mixing is continued until the cyclodextrin is no longer at saturation in the liquid phase of the composition.

8. The process of claim 1, wherein at least 80% by weight of the lipophilic drug and the cyclodextrin in the precipitate are in the combined complexation at the molar ratio of about 1:2, such as at least at least 85%, at least 90%, at least 95% by weight, or at least 99% by weight.

9. The process of claim 1, wherein step d. is carried out in an aqueous alcohol solution.

10. The process of claim 1, wherein the combining and/or mixing steps are carried out at a temperature of between 25° C. to 70° C.

11. The process of claim 1, wherein the cyclodextrin is a native (unmodified) alpha-, beta-, or gamma-cyclodextrin.

12. The process of claim 1, wherein the lipophilic drug has a size between 200 and 600 g/mol; and/or wherein the lipophilic drug has a logP between 3 and 8.

13. (canceled)

14. The process of claim 1, wherein the lipophilic drug is selected from the group consisting of a cannabinoid, Vitamin E or a vitamin E derivative, Vitamin D or a Vitamin D derivative, Vitamin A or a Vitamin A derivative, curcumin or a curcumin derivative, a sterol or a sterol derivative, an antibiotic, a non-steroidal anti-inflammatory (NSAID), and Artemesin or an Artemesin derivative.

15. The process of claim 1, wherein the lipophilic drug is a cannabinoid selected from the group consisting of cannabidiol (CBD), cannabinol (CBN), cannabigerol (CBG) and delta-9-tetrahydrocannabinol (THC).

16. (canceled)

17. A solid composition comprising a lipophilic drug and a cyclodextrin, wherein the lipophilic drug and the cyclodextrin are in the form of a combined complexation and the lipophilic drug and the cyclodextrin in the combined complexation are at a molar ratio of about 1:2,
wherein the cyclodextrin is not a bridged cyclodextrin, and wherein at least 80% by weight of the lipophilic drug and the cyclodextrin in the solid composition are in the combined complexation at the molar ratio of about 1:2.

18. (canceled)

19. The solid composition of claim 17, wherein the cyclodextrin is a native (unmodified) alpha-, beta-, or gamma-cyclodextrin.

20. The solid composition of claim 17, wherein the lipophilic drug has a size between 200 and 600 g/mol, and/or a logP between 3 and 8.

21. (canceled)

22. The solid composition of claim 17, wherein the lipophilic drug is selected from the group consisting of a cannabinoid, Vitamin E or a vitamin E derivative, Vitamin D or a Vitamin D derivative, Vitamin A or a Vitamin A derivative, curcumin or a curcumin derivative, a sterol or a sterol derivative, an antibiotic, a non-steroidal anti-inflammatory (NSAID), and Artemesin or an Artemesin derivative.

23. The solid composition of claim 17, wherein the lipophilic drug is a cannabinoid selected from the group consisting of cannabidiol (CBD), cannabinol (CBN), cannabigerol (CBG) and delta-9-tetrahydrocannabinol (THC).

24. The solid composition of claim 17, wherein the lipophilic drug is the only therapeutically active agent in the solid composition.

25. (canceled)

26. (canceled)

27. A method of treatment of a disease, disorder or condition comprising administering to a subject the solid composition of claim 17 to claim 24.

28. The method of claim 27, wherein the disease, disorder or condition is one associated with the endocannabinoid system, or one associated with a vitamin deficiency (such as a vitamin E deficiency) or one responsive to treatment with an antioxidant (such as curcumin).

29. (canceled)

30. (canceled)

31. The method of claim 27, wherein the solid composition is administered orally, intravenously, subcutaneously, dermally, topically, ophthalmically or by any other parenteral route.

32. The solid composition of claim 17, wherein at least 85%, such as at least 90% by weight, at least 95% by weight, or at least 99% by weight of the lipophilic drug and the cyclodextrin in the solid composition are in the combined complexation at a molar ratio of about 1:2.
